(19) United States
(12) Patent Application Publication
(10) Pub. No.: US 2010/0030322 A1
(43) Pub. Date: Feb. 4, 2010

(54) BRIDGE GRAFT

(76) Inventors: John Sang Hun Lee, Palos Verdes Estates, CA (US); James T. Lee, Huntington Beach, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/273,018

(22) Filed: Nov. 18, 2008

Related U.S. Application Data
(60) Provisional application No. 61/084,953, filed on Jul. 30, 2008.

(51) Int. Cl. A61F 2/06; A61B 19/00

(52) U.S. Cl. 623/1.24; 128/898; 623/1.13

(57) ABSTRACT

An arterio-venous graft includes an implantable tubular member and a flow regulator. The implantable tubular member has a first end and a second end generally opposite the first end. The flow regulator is between the first end and the second end. The flow regulator is configured to regulate fluid flow between the first end and the second end. A method of regulating pressure in an arterio-venous graft includes reversibly adjusting a cross-sectional area of the graft. A method of fluidly coupling an artery and a vein includes connecting a first end of an implantable tubular member to the artery and connecting a second end of the implantable tubular member to the vein. A cross-sectional area of the implantable tubular member is reversibly adjustable.

### CROSS-REFERENCE TO RELATED APPLICATIONS

This application claims priority benefit under 35 U.S.C. §119(e) to U.S. Provisional Patent Application No. 61/084,953, filed Jul. 30, 2008, entitled BRIDGE GRAFT, which is incorporated herein by reference in its entirety.

### BACKGROUND

1. Field

The present application relates generally to bridge grafts and the implantation and usage thereof More particularly, the present application relates to bridge grafts for use in dialysis (e.g., hemodialysis), filtration (e.g., ultrafiltration), and pheresis (e.g., plasmapheresis).

2. Description of the Related Art

In 2004, there were approximately 472,000 patients with end-stage renal disease (ESRD) in the United States. The projected ESRD population by the year 2010 is estimated to be greater than 650,000. Medicare costs in 2004 for treating ESRD patients were $32.5 billion, or approximately 7.2% of the Medicare budget. Non-Medicare costs that same year were approximately $12.4 billion, which represents an increase of 57% versus 1999 non-Medicare costs. In 2004, modalities employed for patients with ESRD included hemodialysis (HD) (approximately 65.6%), renal transplant (about 28.9%), and peritoneal dialysis (PD) (less than about 5.5%). Accordingly, hemodialysis is the most commonly used procedure for ESRD patients, the population of which is growing every year.

A static bridge graft (i.e., with no moving parts) may be installed between an ERSD patient's artery and vein such that a dialysis machine can access a blood supply through the graft. Dialysis machines replicate the function of the diseased kidneys, so they generally require the circulation of large volumes of blood in order to remove waste from the blood. Thus, static devices are typically configured to continuously provide a maximum amount of flow through the graft.

### SUMMARY

Certain arterio-venous bridge grafts disclosed herein comprise a flow regulator that can decrease venous barotrauma by decreasing pressure. By decreasing the cross-sectional area (e.g., luminal diameter) of the graft, the pressure through the graft decreases in accordance with Bernoulli's principle, thus transmitting less arterial pressure to the venous outflow and the venous anastamosis. In certain embodiments, this feature is reversible to provide a circuit having high fluid flow and fluid pressure during dialysis or other treatments. In some embodiments, a self-expanding covered stent is disposed in an implantable tubular member. In certain such embodiments, the stent can be at least partially constrained between treatments, and the expansionary properties of the stent can be used to reverse the mechanism and allow expansion of the graft during treatments. In certain embodiments, the flow regulator comprises a mechanical switch or an electrical switch. In some embodiments, the flow regulator comprises a protective sleeve to reduce (e.g., minimize, prevent) tissue ingrowth, which could occlude the stent and render the stent inoperable. In certain embodiments, the flow regulator may be designed so that a high pressure endovascular balloon angioplasty can be used to restore the stent to patency if the stent fails to expand from the constrained position.

In certain embodiments, a method of regulating pressure in an arterio-venous bridge graft comprises reversibly adjusting a cross-sectional area of the graft.

In certain embodiments, a method of fluidly coupling an artery and a vein comprises connecting a first end of an implantable tubular member to the artery, and connecting a second end of the implantable tubular member to the vein, the second end generally opposite the first end, a cross-sectional area of the implantable tubular member being reversibly adjustable.

In certain embodiments, an arterio-venous graft comprises an implantable tubular member having a first end and a second end generally opposite the first end, and a flow regulator between the first end and the second end, the flow regulator configured to reversibly regulate fluid flow between the first end and the second end.

For purposes of summarizing the invention and the advantages achieved over the prior art, certain objects and advantages of the invention are described herein. Of course, it is to be understood that not necessarily all such objects or advantages may be achieved in accordance with any particular embodiment. Thus, for example, those skilled in the art will recognize that the invention may be embodied or carried out in a manner that achieves or optimizes one advantage or group of advantages as taught or suggested herein without necessarily achieving other objects or advantages as may be taught or suggested herein.

All of these embodiments are intended to be within the scope of the invention herein disclosed. These and other embodiments will become readily apparent to those skilled in the art from the following detailed description having reference to the attached figures, the invention not being limited to any particular preferred embodiment(s) disclosed.

### BRIEF DESCRIPTION OF THE DRAWINGS

These and other features, aspects, and advantages of the invention disclosed herein are described below with reference to the drawings of certain embodiments, which are intended to illustrate and not to limit the invention.

FIG. 1 schematically illustrates an example embodiment of an arterio-venous bridge graft.

FIG. 2A is a schematic perspective view of an example embodiment of an arterio-venous bridge graft in a first state.

FIG. 2B is a cross-section of the bridge graft of FIG. 2A.

FIG. 3A schematically illustrates the bridge graft of FIG. 2A in a second state.

FIG. 3B is a cross-section of the bridge graft of FIG. 3A.

FIG. 4A is a schematic perspective view of another example embodiment of an arterio-venous bridge graft in a first state.

FIG. 4B is a cross-section of the bridge graft of FIG. 4A.

FIG. 5A schematically illustrates the bridge graft of FIG. 4A in a second state.

FIG. 5B is a cross-section of the bridge graft of FIG. 5A.

FIG. 6A is a schematic perspective view of a still another example embodiment of an arterio-venous bridge graft in a first state.

FIG. 6B is a cross-section of the bridge graft of FIG. 6A.

FIG. 7A schematically illustrates the bridge graft of FIG. 6A in a second state.

FIG. 7B is a cross-section of the bridge graft of FIG. 7A.

FIG. 8A is a schematic perspective view of yet another example embodiment of an arterio-venous bridge graft in a first state.

FIG. 8B is a cross-section of the bridge graft of FIG. 8A.

FIG. 9A schematically illustrates the bridge graft of FIG. 8A in a second state.

FIG. 9B is a cross-section of the bridge graft of FIG. 9A.

FIG. 10A schematically illustrates an example embodiment of a method of fluidly coupling an artery and a vein for dialysis treatment.

FIG. 10B schematically illustrates another example embodiment of a method of fluidly coupling an artery and a vein for dialysis treatment.

FIG. 11 schematically illustrates an example embodiment of a dynamic bridge graft installed in a patient.

FIG. 12A schematically illustrates an example of a static bridge graft installed in a patient.

FIG. 12B schematically illustrates an example embodiment of a dynamic bridge graft installed in a patient.

FIG. 13A schematically illustrates an example of a static bridge graft installed in a patient.

FIG. 13B schematically illustrates an example embodiment of a dynamic bridge graft installed in a patient.

### DETAILED DESCRIPTION

Although certain embodiments and examples are disclosed below, it will be understood by those in the art that the invention extends beyond the specifically disclosed embodiments and/or uses of the invention and obvious modifications and equivalents thereof Thus, it is intended that the scope of the invention herein disclosed should not be limited by the particular embodiments described below.

The majority of ERSD patients receive an arterio-venous bridge graft between a native artery and a native vein in the upper or lower portion of an appendage. For example, the bridge graft may be installed in either arm below or above the elbow or in either leg above or below the knee. The graft is preferably first installed below the elbow in the non-dominant arm. Once the bridge graft is installed, the access needles of a dialysis machine may be inserted into the bridge graft to provide access to a flow of blood, which the dialysis machine can purify and return to the body.

Hemodialysis is performed on average during three visits per week for an average duration of about three to about four hours per visit. The graft is therefore in use for approximately nine to twelve hours per week. This is the only period during which high fluid flow and fluid pressure in the graft is desirable. However, complications in the bridge graft may reduce the effectiveness of the treatment or may cause issues with the patient's extremities or elsewhere. Continuous high pressure can negatively impact the inner lining of a native vein in the form of venous barotrauma, which can cause, inter alia, pain, venous irritation, scarring, stenosis, necrosis, limb loss, occlusion, pallor, imprecise blood pressure measurements, increased cardiac demands, steal syndrome, ischemia, myointimal hyperplasia, and/or pseudoaneurysm formation. For example, about 85% of all bridge graft complications may be the result of thrombosis caused by myointimal hyperplasia buildup at the venous anastamosis. This occurs in up to 90% of ERSD patients with a prosthetic graft (i.e., not grafts from other portions of the body or from other people or animals), and the one year patency rate after intervention can range from about 3% to about 36%.

In accordance with certain embodiments described herein, Applicants have realized that by limiting the duration of the high pressure and high flow on the vein, the patency rate of the graft can be significantly improved, thereby reducing (e.g., minimizing, eliminating) the need for secondary interventions, which can reduce (e.g., minimize, eliminate) associated complications and/or reduce (e.g., minimize, eliminate, exponentially reduce) healthcare expenditures associated with post intervention patency. The bridge grafts described herein can thus be used for “First Line” access for all patients requiring hemodialysis. Reducing venous barotrauma can also reduce (e.g., minimize, eliminate) pain, venous irritation, scarring, stenosis, necrosis, limb loss, occlusion, pallor, imprecise blood pressure measurements, increased cardiac demands, steal syndrome, ischemia, myointimal hyperplasia, pseudoaneurysm formation, and/or other adverse effects that may be associated with bridge grafts providing continuous high pressure.

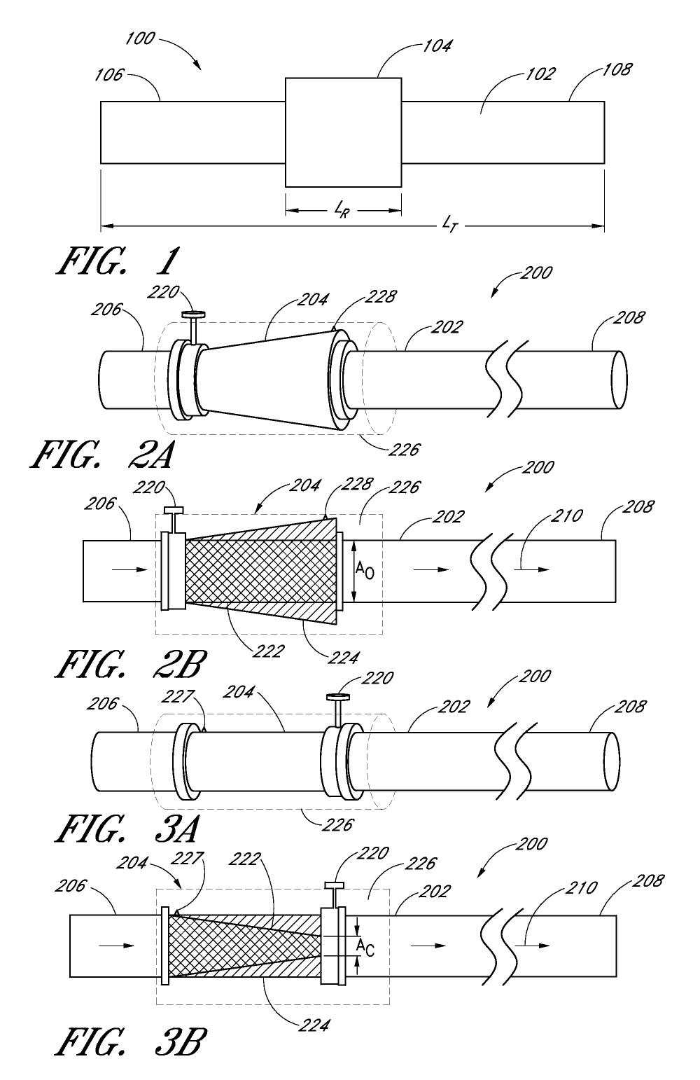

FIG. 1 schematically illustrates an example embodiment of an arterio-venous bridge graft 100 that can be used to access a blood supply during a treatment such as dialysis (e.g., hemodialysis), filtration (e.g., ultrafiltration), and pheresis (e.g., plasmapheresis). The graft 100 may also be used for artery-artery and vein-vein implantations. The graft 100 comprises an implantable tubular member 102 having a first end 106 and a second end 108 generally opposite a first end 106. The implantable tubular member 102 defines a lumen. The graft 100 further comprises a flow regulator 104 between the first end 106 and the second end 108 of the implantable tubular member 102. The flow regulator 104 is configured to regulate fluid flow between the first end 106 and the second end 108. The graft 100 may therefore be characterized as a dynamic bridge graft. The dimensions (e.g., diameter) of the graft 100 may be closely associated with, but are not limited to, the dimensions (e.g., diameter) of the native donor artery in order to promote laminar flow at the arterial anastamosis.

In some embodiments, the implantable tubular member 102 comprises a synthetic material (e.g., polytetrafluoroethylene (PTFE), Dacron). In certain alternative embodiments, the implantable tubular member 102 comprises a natural material (e.g., an artery or a vein taken from another part of the patient's body or a donor human or animal). In some embodiments, the implantable tubular member 102 is flexible. Each of the components of the graft 100 preferably comprises a biocompatible material. In certain embodiments, the first end 106 is configured to be fluidly coupled to an artery (e.g., via arterial anastamosis) and the second end 108 is configured to be fluidly coupled to a vein (e.g., via venous anastamosis). In certain alternative embodiments, the first end 106 is configured to be fluidly coupled to a vein (e.g., via venous anastamosis) and the second end 108 is configured to be fluidly coupled to a second vein (e.g., via second venous anastamosis).

The implantable tubular member 102 has a length L_{T} between the first end 106 and the second end 108, and the flow regulator 104 has a length L_{R} extending along the longitudinal axis of the implantable tubular member 104. The length L_{R} of the flow regulator may be designed or selected based on the projected location of implantation into the patient, age of the patient, size of the patient, cross-sectional area of the graft 100, cross-sectional area of the upstream artery or vein, cross-sectional area of the downstream artery or vein, length L_{T} of the graft 100, the type of flow regulator 104, result of an Allen's test, patient comorbidity, combinations thereof, and the like. In certain embodiments, the length L_{R} of the flow regulator 104 is less than about ⅓ of the length L_{T} of the implantable tubular member 102 (i.e., L_{R}<L_{T}/3). The lengths L_{T} and L_{R} and other properties of the graft 100 may also influenced by bench and animal models.

In certain embodiments, the flow regulator 104 comprises a valve configured to increase fluid flow through the implantable tubular member 102 during a treatment (e.g., hemodialysis). In some embodiments, the flow regulator 104 comprises a mechanical switch configured to increase fluid flow through the implantable tubular member 102 during a treatment (e.g., hemodialysis). In some embodiments, the flow regulator 104 comprises a cylindrical flow limiter configured to increase fluid flow through the implantable tubular member 102 during a treatment (e.g., hemodialysis). In some embodiments, the flow regulator 104 comprises an electrical switch configured to increase fluid flow through the implantable tubular member 102 during a treatment (e.g., hemodialysis). In certain such embodiments, the flow regulator 104 comprises a timer configured to operate the switch and/or a sensor configured to operate the switch upon a change in a parameter of the fluid flow to increase fluid flow through the implantable tubular member 102 during a treatment (e.g., hemodialysis). The parameter may include fluid flow rate, fluid velocity, fluid pressure, combinations thereof, and the like. For example, if a velocity sensor indicates that a clot may be forming (e.g., because velocity is reduced below a certain level), then the switch may be operated to at least partially open the flow regulator. In certain embodiments, a timer can be configured to cycle dilation and constriction of the flow regulator 104 to reduce (e.g., minimize, eliminate) thrombosis (e.g., independent of any treatments). For example, the timer may be programmed to cycle to an at least partially open state at certain intervals (e.g., based on an average clot time). In certain embodiments, a sensor can be configured to increase or decrease fluid flow through the flow regulator 104 upon a change in a parameter of the fluid flow (e.g., fluid flow rate, fluid velocity, fluid pressure) to reduce (e.g., minimize, eliminate) thrombosis (e.g., independent of any treatments).

In some embodiments, the flow regulator 104 comprises a self-expanding stent (e.g., comprising a shape memory alloy (e.g., nitinol)) at least partially, substantially, or fully covered and/or lined by a material configured to restrain fluid flow (e.g., PTFE). In certain such embodiments, the flow regulator 104 may be configured to be disposed between the first end 106 and the second end 108 of a PTFE implantable tubular member 102 and configured to regulate fluid flow between the first end 106 and the second end 108. Other flow regulators 104 are also possible. The flow regulator 104 may optionally be manufactured separately and later joined to the implantable tubular member 102.

In some embodiments, the implantable tubular member 102 comprises two discrete pieces that are each fluidly coupled to the flow regulator 104. In certain such embodiments, the lumens of the pieces of the implantable tubular member 102 are preferably aligned. In some embodiments, the flow regulator 104 is disposed within or around a continuous implantable tubular member 102. For example, a stent (e.g., a self-expanding stent) may be coupled to the outside of a PTFE tube. For another example, a stent (e.g., a self-expanding stent) may be coupled to the inside of a PTFE tube. In some embodiments, the implantable tubular member 102 and the flow regulator 104 are integrated as a single continuous piece. For example, in embodiments in which the implantable tubular member 102 is molded, a stent may be disposed in the mold and the implantable tubular member 102 may be formed above and/or below the stent to form the flow regulator 104. In certain such embodiments, the material of the implantable tubular member 102 preferably does not inhibit operation of the flow regulator 104.

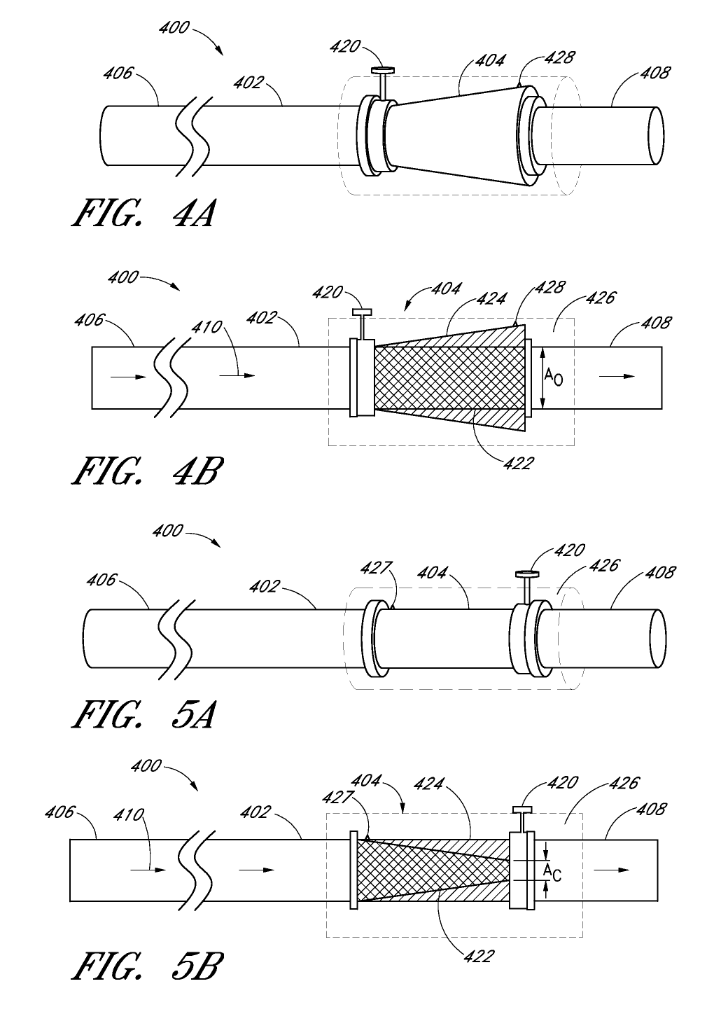

FIG. 2A is a schematic perspective view of an example embodiment of an arterio-venous bridge graft 200 that can be used to access a blood supply during a treatment (e.g., hemodialysis). The graft 200 comprises an implantable tubular member 202 and a flow regulator 204. The implantable tubular member 200 has a first end 206 and a second end 208 generally opposite the first end 206. The implantable tubular member 202 defines a lumen. The flow regulator 204 is between the first end 206 and the second end 208. The flow regulator 204 is configured to regulate fluid flow between the first end 206 and the second end 208. The flow regulator 204 is proximate the first end 206 of the implantable tubular member 202. In certain such embodiments, the flow regulator 204 is close enough to the first end 206 that there is substantially laminar flow and reduced stagnation, but the flow regulator 204 is far enough from the first end 206 to allow safe attachment to an artery or vein. In some embodiments, the flow regulator 204 is less than about 10 cm, less than about 5 cm, or less than about 2 cm from the first end 206.

FIG. 2B illustrates a cross-sectional view of the graft 200 of FIG. 2A taken along the longitudinal axis of the implantable tubular member 202. The graft 200 is configured so that fluid flows through the lumen defined by the implantable tubular member 202 in the direction indicated by the arrows 210. In alternative embodiments, the graft 200 may be configured so that fluid flows through the lumen defined by the implantable tubular member 202 in the direction opposite to the direction indicated by the arrows 210.

The flow regulator 204 in the embodiment illustrated in FIG. 2A comprises a hollow member 220 (e.g., a ring) surrounding (e.g., partially surrounding, substantially surrounding) a self-expanding stent 222. FIGS. 2A and 2B illustrate the flow regulator 204 in a first state (e.g., in which the hollow member 220 is closer to the first end 206 than the second end 208; substantially open). FIGS. 3A and 3B illustrate, in perspective view and in cross-sectional view, respectively, the graft 200 in a second state (e.g., in which the hollow member 220 is closer to the second end 208 than the first end 206; at least partially constricted), for example after the hollow member 220 has been manipulated. The stent 222 is substantially fully open (e.g., having a cross-sectional area A_{O} that is substantially similar or equal to the cross-sectional area of the lumen defined by the implantable tubular member 202) in the first state and is at least partially constricted in the second state (e.g., having a cross-sectional area A_{C} that is less than the cross-sectional area of the lumen defined by the implantable tubular member 202). In certain embodiments, the flow regulator reduces pressure through the implantable tubular member 202 enough to reduce (e.g., minimize, prevent) venous hyperplasia. In some embodiments, the flow regulator 204 is configured to reduce fluid flow through the implantable tubular member 202 by at least about 60% (i.e., A_{C}≦0.4×A_{O}). In some embodiments, the flow regulator 204 is configured to reduce fluid flow through the implantable tubular member 202 by at less than about 90% (i.e., A_{C}≦0.9×A_{O}). In some embodiments, the flow regulator 204 is configured to increase fluid flow through the implantable tubular member 202 by at least about 150% (i.e., A_{O}≧2.5×A_{C}). In certain embodiments, the flow regulator 204 comprises a sleeve 226 configured to prevent tissue ingrowth, to increase durability of the flow regulator, and/or to facilitate operation of the flow regulator 204. For example, the sleeve 226 may comprise a sterile biocompatible material (e.g., PTFE, Dacron, plastic, silicone, metal such as stainless steel, nitinol, etc.). In some embodiments, the sleeve extends over at least one junction between the implantable tubular member 202 and the flow regulator 204. In some embodiments, the amount of flow reduction (A_{C}/A_{O} ratio) in the flow regulator 204 is determined based on bench and animal models and is adjusted to achieve a desired effect that can reduce (e.g., minimize, eliminate) certain complications associated with bridge grafts (e.g., venous barotrauma, steal syndrome, pseudoaneurysm formation, etc.), but that can also maintain graft patency.

A cross-sectional area of the stent 222 is configured to decrease from A_{O} to A_{C} upon movement of the hollow member 220 from a first position (e.g., closer to the first end 206 than to the second end 208, as illustrated in FIGS. 2A and 2B) to a second position (e.g., closer to the second end 208 than to the first end 206, as illustrated in FIGS. 3A and 3B) and is configured to increase from A_{C} to A_{O} upon movement of the hollow member from the second position to the first position. FIGS. 2A through 3B illustrate an example embodiment of a graft 200 comprising a flow regulator 204 comprising a stent 222, although other types of flow regulators 204 are also possible.

The illustrated flow regulator 204 comprises bearing surfaces 224 in contact with the stent 222. In certain embodiments, the bearing surfaces 224 are in mechanical communication with the stent 222 (e.g., through a PTFE coating). In some embodiments, the bearing surfaces 224 comprise one or more tapered projections (e.g., flares, wings) extending outwardly from the stent 222. The bearing surfaces 224 are less prone to deformation upon the application of a force than the stent 222 or the hollow member 220. For example, the bearing surfaces 224 may comprise plastic, silicone, or metal such as stainless steel, nitinol, etc. As the hollow member 220 moves from the left to the right in the Figures, the bearing surfaces 224 are inwardly displaced, thereby crimping or collapsing the stent 222. The bearing surfaces 224 may be disposed symmetrically around the stent 222 (e.g., as illustrated in FIGS. 2B and 3B) or may be disposed asymmetrically around the stent 222. In some embodiments, the constricted cross-sectional area A_{C} may be approximated by subtracting the width of the widest portions of the bearing surfaces 224 from the open area A_{O}. As the hollow member 220 moves from the right to the left in the Figures, the bearing surfaces 224 may be outwardly displaced by the stent 222, thereby opening the stent 222. In certain embodiments, the stent 222 is self-expanding. In some embodiments, the flow regulator 204 comprises a feature (e.g., a nub 227, 228 at one or both ends of the flow regulator 204) configured to maintain the position of the hollow member 220. The illustrated flow regulator 204 is just an example embodiment of flow regulator. Other flow regulators, for example that can be electronically and/or mechanically operated, are also possible.

In some embodiments, the flow regulator 204 may be mechanically manipulated by applying force to a member (e.g., the hollow member 220 or a portion thereof) disposed proximate to or extending through the epidermis. For example, the member may be a subdermal bump that can be grasped by a hand or a tool and slid or turned relative to the implantable tubular member. In some embodiments, the flow regulator 204 may be electrically manipulated by applying a current to operate an electronic motor connected to a valve. In certain such embodiments, the graft 200 may comprise a battery. In some embodiments, the flow regulator 204 may be magnetically manipulated by applying a magnetic field to effect movement of a member or a valve. In some embodiments, the flow regulator 204 may be operated via remote control (e.g., using radio frequencies, Bluetooth, or the like). In certain embodiments, the graft comprises one or more radio opaque markers that allow detection of position. As an example, the hollow member 220 and the nubs 227, 228 may comprise radio opaque markers.

FIGS. 4A through 5B illustrate another example embodiment of a graft 400 that can be used to access a blood supply during a treatment (e.g., hemodialysis). The graft 400 comprises an implantable tubular member 402 and a flow regulator 404. The implantable tubular member 400 has a first end 406 and a second end 408 generally opposite the first end 406. The implantable tubular member 402 defines a lumen. The flow regulator 404 is between the first end 406 and the second end 408. The flow regulator 404 is configured to regulate fluid flow between the first end 406 and the second end 408. The flow regulator 404, which is illustrated as being similar to the flow regulator 204 illustrated in FIGS. 2A through 3B, is proximate the second end 408 of the implantable tubular member 402. In certain such embodiments, the flow regulator 404 is close enough to the second end 408 that there is substantially laminar flow and reduced stagnation, but the flow regulator 404 is far enough from the second end 408 to allow safe attachment to an artery or vein. In some embodiments, the flow regulator 404 is less than about 10 cm, less than about 5 cm, or less than about 2 cm from the second end 408. The flow regulator 204, 404 may be disposed anywhere along the implantable tubular member 202, 402, although proximity to one end or another may be useful for certain applications. For example, the position of the flow regulator 204, 404 may be designed based on the projected implant location in a patient, age of the patient, size of the patient, cross-sectional area of the graft 200, 400, cross-sectional area of the upstream artery or vein, cross-sectional area of the downstream artery or vein, length L_{T} of the graft 200, 400, the type of flow regulator 204, 404, combinations thereof, and the like. If a patient has a positive Allen's Test, which can indicate poor circulation to the extremity, the flow regulator 204, 404 may be disposed on the end of the graft 200, 400 configured to be connected to the artery. If a patient has a negative Allen's Test, which can indicate good circulation to the extremity, the flow regulator 204, 404 may be disposed on the end of the graft 200, 400 configured to be connected to the vein. Other patient comorbidity may also influence the position of the flow regulator 204, 404. The ability to modify the location of the flow regulator 202, 404 can advantageously allow use in patients with pathologic microvascular circulatory disease.

In the embodiments illustrated in FIGS. 2A through 5B, the first end 206, 406 has a first cross-sectional area and the second end 208, 408 has a second cross-sectional area. The first cross-sectional area is substantially equal to the second cross-sectional area. In certain such embodiments, the cross-sectional area of the first end 206, 406 is between about 28 millimeters squared (mm²) and about 29 mm², and the cross-sectional area of the second end is between about 28 mm² and about 29 mm². Other cross-sectional areas are also possible. In some embodiments, the implantable tubular member 202, 402 has a substantially uniform inner diameter. It will be appreciated that in embodiments in which the implantable tubular member 202 comprises a cylindrical shape or a substantially cylindrical shape, the diameter of the implantable tubular member 202 may be in the range of about 4 mm to about 7 mm (e.g., about 6 mm). Other shapes and diameters are also possible.

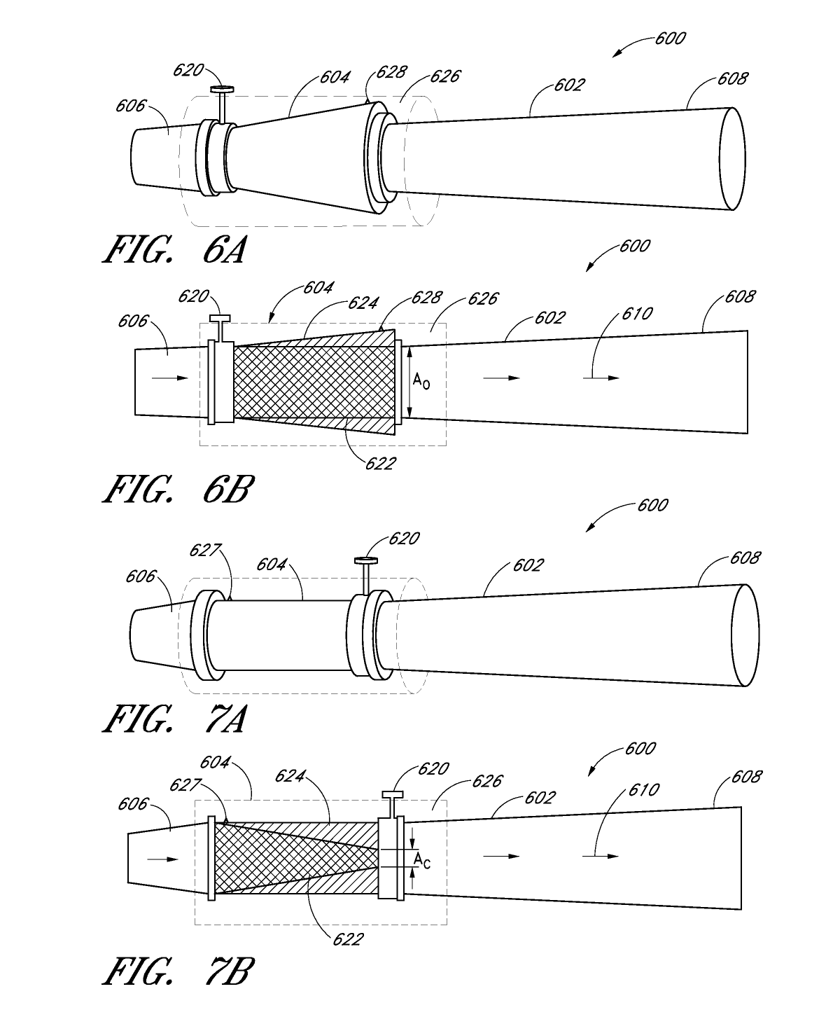

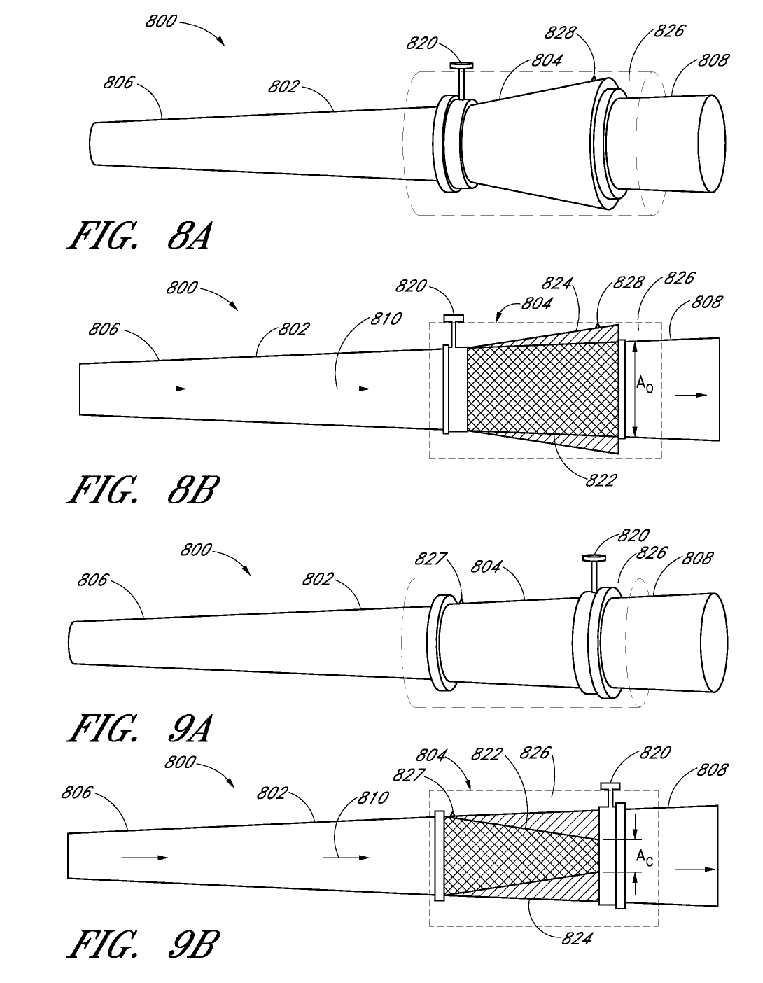

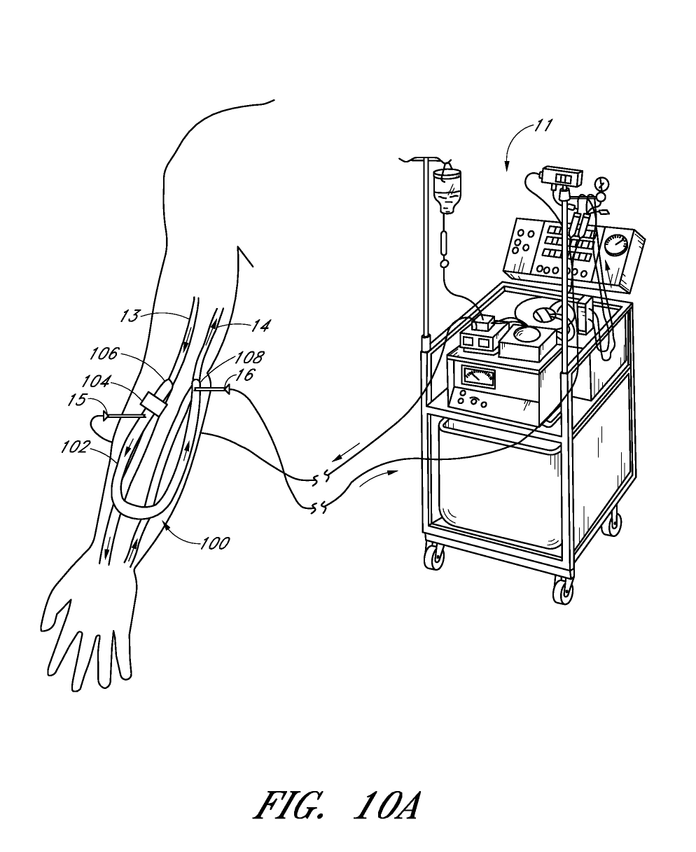

FIGS. 6A through 7B illustrate still another example embodiment of an arterio-venous bridge graft 600 that can be used to access a blood supply during a treatment (e.g., hemodialysis). The graft 600 comprises an implantable tubular member 602 and a flow regulator 604. The implantable tubular member 600 has a first end 606 and a second end 608 generally opposite the first end 606. The implantable tubular member 602 defines a lumen. The flow regulator 604 is between the first end 606 and the flared second end 608. The flow regulator 604 is configured to regulate fluid flow between the first end 606 and the second end 608. The flow regulator 604, which is illustrated as being similar to the flow regulator 204 illustrated in FIGS. 2A through 3B, is proximate the first end 606 of the implantable tubular member 602.

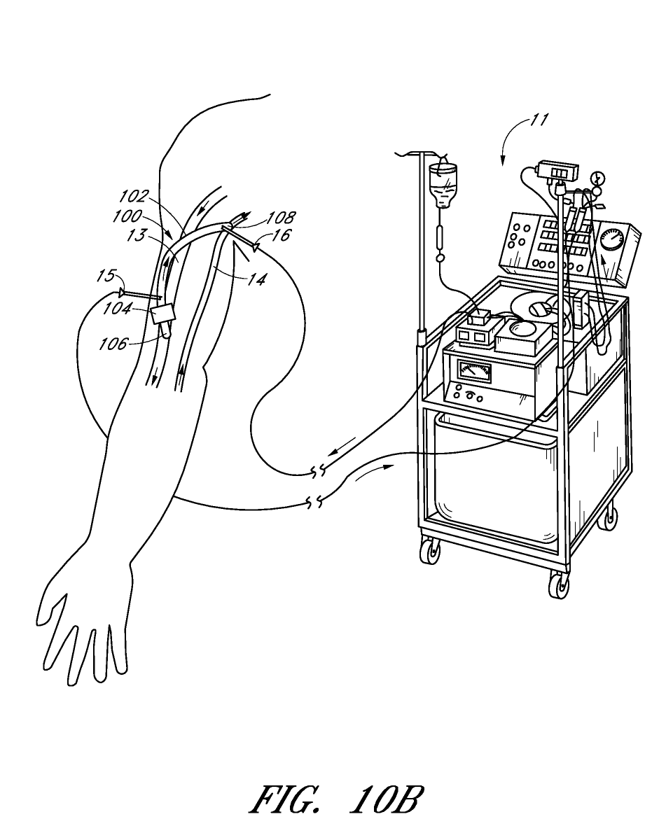

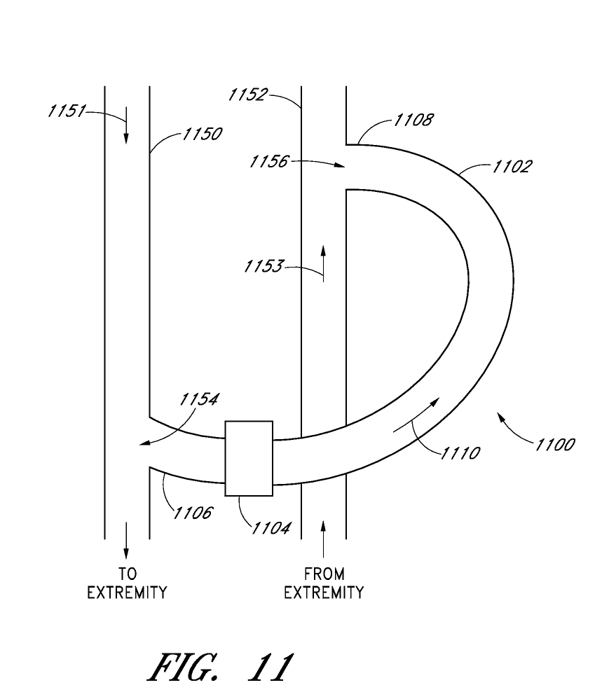

FIGS. 8A through 9B illustrate yet another example embodiment of an arterio-venous bridge graft 800 that can be used to access a blood supply during a treatment (e.g., hemodialysis). The graft 800 comprises an implantable tubular member 802 and a flow regulator 804. The implantable tubular member 800 has a first end 806 and a second end 808 generally opposite the first end 806. The implantable tubular member 802 defines a lumen. The flow regulator 804 is between the first end 806 and the flared second end 808. The flow regulator 804 is configured to regulate fluid flow between the first end 806 and the second end 808. The flow regulator 804, which is illustrated as being similar to the flow regulator 204 illustrated in FIGS. 4A through 5B, is proximate the second end 808 of the implantable tubular member 602.

In the embodiments illustrated in FIG. 6A through 9B, the first end 606, 806 has a first cross-sectional area and the second end 608, 808 has a second cross-sectional area greater than the first cross-sectional area. The implantable tubular member 602, 802 has a cross-section generally increasing from the first end 606, 806 to the second end 608, 808. In certain such embodiments, the first end 606, 806 is configured to be fluidly coupled to an artery (e.g., via arterial anastamosis). In some embodiments, the cross-sectional area of the first end 606, 806 is between about 12 mm² and about 13 mm² and the cross-sectional area of the second end 608, 808 is between about 38 mm² and about 39 mm². In embodiments in which the implantable tubular member 602, 802 comprises a tapered tube, the diameter at the first end 606, 806 may be in the range of about 3 mm to about 5 mm (e.g., about 4 mm) and the diameter at the second end 608, 808 may be in the range of about 5 mm to about 8 mm (e.g., about 7 mm). Other shapes and diameters are also possible.

In certain embodiments, the implantable tubular member 602, 802 has the same cross-sectional area (e.g., diameter) on each side of the flow regulator 604, 804 such that the taper is interrupted by the flow regulator 604, 804 (e.g., as illustrated in FIGS. 6A through 7B). In certain alternative embodiments, the implantable tubular member 602, 802 has a different cross-sectional area (e.g., diameter) on each side of the flow regulator 604, 804 such that the taper continued through the flow regulator 604, 804 (e.g., as illustrated in FIGS. 8A through 9B). In certain such embodiments, the stent 622, 822 may be tapered in an open position.

The grafts described herein may be designed or selected for a particular patient. For example, the flow regulator may be disposed anywhere along the implantable tubular member. For another example, the implantable tubular member may be tapered towards one end. For yet another example, the length of a transition zone defined by the flow regulator may be increased or decreased. Some considerations for design or selection include the projected location of implantation into the patient, age of the patient, size of the patient, cross-sectional area of the upstream artery or vein, cross-sectional area of the downstream artery or vein, the type of flow regulator, result of an Allen's test, patient comorbidity, combinations thereof, and the like.

Although FIGS. 2A through 9B illustrate arterio-venous bridge grafts in various states (e.g., open, at least partially constricted), having flow regulators in various positions (e.g., proximate to the first end, proximate to the second end), and having different shapes (e.g., tubular, tapered), any combination of the features illustrated therein is also possible. Additionally, although the flow regulators are illustrated as comprising a stent, other types of flow regulators are also possible.

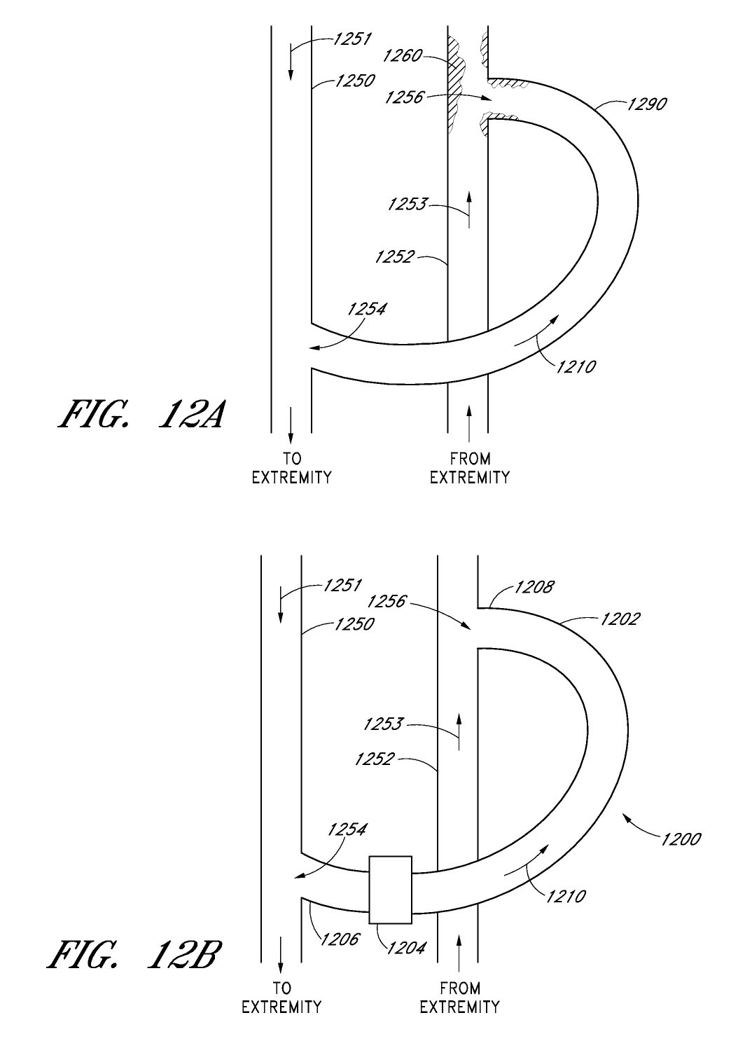

FIG. 10A illustrates a method of fluidly coupling an artery 13 and a vein 14. The method comprises connecting a first end 106 of an implantable tubular member 102 to the artery 13 (e.g., via arterial anastamosis) and connecting a second end 108 of the implantable tubular member 102, which is generally opposite the first end 106, to the vein 14 (e.g., via venous anastamosis). The implantable tubular member 102 defines a lumen. A flow regulator 104 is disposed between the first end 106 and the second end 108. The flow regulator 104 is configured to regulate fluid flow between the first end 106 and the second end 108. Although illustrated as being installed below the elbow in a patient's right forearm (e.g., as illustrated by FIG. 10B), the graft 100 may be implanted above the elbow, in the left forearm, above the left elbow, and in the legs. In certain embodiments in which the implantable tubular member 102 comprises a tapered tube (e.g., as illustrated in FIGS. 6A through 9B), the first end 106 has a cross-sectional area greater than the second end 108. In certain alternative embodiments in which the implantable tubular member 102 comprises a tapered tube (e.g., as illustrated in FIGS. 6A through 9B), the first end 106 has a cross-sectional area less than the second end 108.

During a treatment (e.g., hemodialysis), the flow regulator 104 may be operated a plurality of times (e.g., twice) to adjust a cross-sectional area of the graft 100. In the first operation of the flow regulator 104, the cross-sectional area of the graft 100 is increased (e.g., to A_{O}) to allow blood to flow through the graft 100 at high pressure. Access needles 15, 16, which are fluidly coupled to a dialysis machine 11, are inserted through the skin and through the wall of the implantable tubular member 102, thereby providing a path for blood to flow from the patient's body into the dialysis machine 11. Increasing the amount of blood flowing through the graft 100 during dialysis can increase (e.g., maximize) therapeutic benefits, for example reduced treatment duration and/or reduced treatment frequency. A countervailing concern is that allowing too much blood to flow through the graft 100 during dialysis can lead to heart failure.

Although the needles 15, 16 are illustrated as being inserted proximate to the first end 106 and the second end 108, the needles 15, 16 may also be inserted more distal to the first end 106 and more proximal to the second end 108. Additionally, the needles 15, 16 may both be inserted proximal to the flow regulator 104, both distal to the flow regulator 104 (e.g., as illustrated in FIG. 10A), or one proximal to the flow regulator 104 and one distal to the flow regulator 104. The needles 15, 16 may be inserted before or after the first operation of the flow regulator 104. In some embodiments in which the flow regulator 104 comprises a self-expanding stent substantially covered and/or lined by PTFE, the first operation comprises allowing expansion of the stent (e.g., by manipulating a hollow member as described above with respect to FIGS. 2A through 9B). In some embodiments, operating the flow regulator 104 may comprise operating a switch. As an example, in embodiments in which the flow regulator 104 comprises a timer, a change in time (e.g., a duration after the inception of a predetermined sequence) may trigger operation of the switch. As another example, in embodiments in which the flow regulator 104 comprises a sensor, a change in a parameter (e.g., fluid flow rate, fluid velocity, fluid pressure) may trigger operation of the switch.

The machine 11 withdraws blood from the artery 13 and removes waste products (e.g., urea) from blood, then reintroduces the blood to the vein 14 through the needle 16 in the implantable tubular member 102. After the machine 11 has cleansed the blood (e.g., after about 3 to 4 hours), the flow regulator 104 is operated a second time. In the second operation of the flow regulator 104, the cross-sectional area of the graft 100 is reduced (e.g., to A_{C}) to allow blood to flow through the graft 100 at low (e.g., less than arterial) pressure. Access needles 15, 16 are removed from the implantable tubular member 102. The needles 15, 16 may be removed before or after the second operation of the flow regulator 104. In some embodiments in which the flow regulator 104 comprises a self-expanding stent substantially covered and/or lined by PTFE, the second operation comprises crimping the stent (e.g., by manipulating a hollow member as described above with respect to FIGS. 2A through 9B). In some embodiments, operating the flow regulator 104 may comprise operating a switch. As an example, in embodiments in which the flow regulator 104 comprises a timer, a change in time (e.g., a duration after the inception of a predetermined sequence) may trigger operation of the switch. As another example, in embodiments in which the flow regulator 104 comprises a sensor, a change in a parameter (e.g., fluid flow rate, fluid velocity, fluid pressure) may trigger operation of the switch.

FIG. 11 illustrates an expanded view of an arterio-venous bridge graft 1100 implanted into a patient, regardless of position on the patient's body. The graft 1100 comprises an implantable tubular member 1102 and a flow regulator 1104. The implantable tubular member 1100 has a first end 1106 and a second end 1108 generally opposite the first end 1106. The implantable tubular member 1102 defines a lumen. The flow regulator 1104 is between the first end 1106 and the second end 1108. The flow regulator 1104 is configured to regulate fluid flow between the first end 1106 and the second end 1108. The first end 1106 is fluidly coupled to an artery 1150 at position 1154 (e.g., via arterial anastamosis). Blood flows through the artery 1150 in the direction indicated by the arrow 1151. In the area 1154, some blood is diverted into the graft 1100, and the rest of the blood flows to an extremity (e.g., a hand, a foot). The second end 1108 is fluidly coupled to a vein 1152 at position 1156 (e.g., via venous anastamosis). Blood flows through the vein 1152 in the direction indicated by the arrow 1153. In the area 1156, some blood returns from the extremity and joins blood that flowed through the graft in the direction indicated by the arrow 1110.

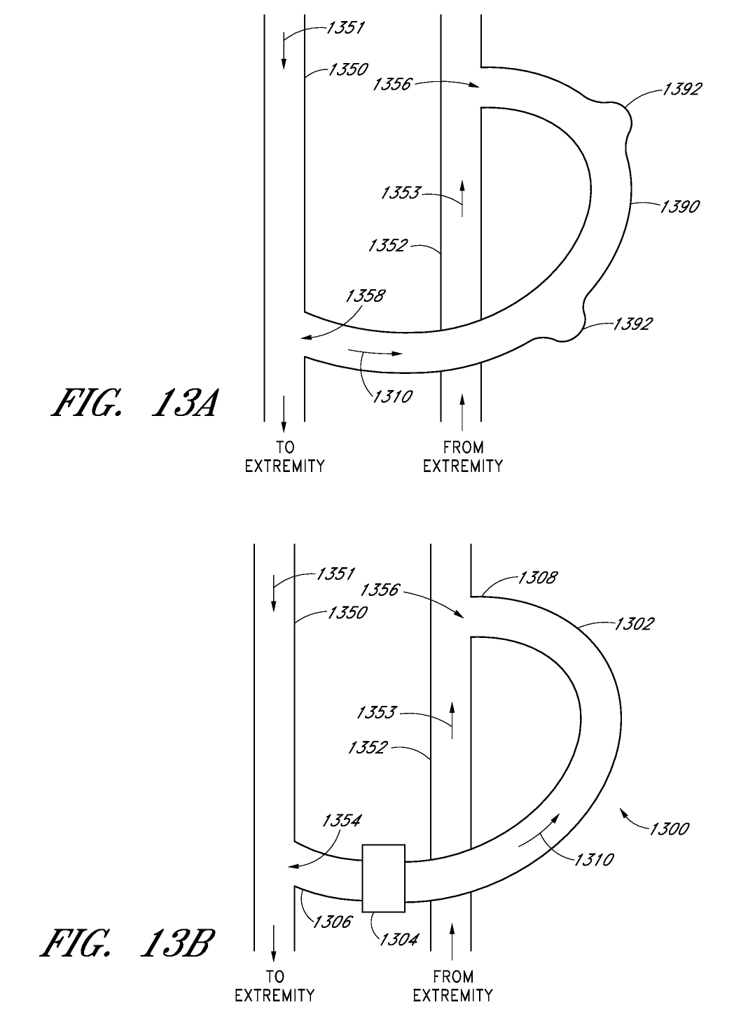

FIG. 12A illustrates a static graft 1290 implanted between an artery 1250 and a vein 1252. The graft 1290 allows continued high pressure fluid flow into the vein 1252. Over time, the high pressure of the blood from the artery 1250 can cause venous barotrauma, which can cause venous irritation and scarring, which can cause thrombosis 1260 to accumulate proximate to the venous anastamosis in the area 1256, possibly leading to stenosis, thrombosis, and/or occlusion. The solution to this problem is usually to revise the graft 1290, to attempt multiple thrombectomu intervensions, and/or to implant a new graft 1290 elsewhere in the patient's body (e.g., a different location on the appendage, a different appendage). Grafts 1290 tapered outward at the venous end have been ineffective because the quantity of blood flowing downstream of the venous anastamosis may still cause barotraumas, distal tissue necrosis, and/or increased cardiac demands.

FIG. 12B illustrates an arterio-venous bridge graft 1200 comprising a flow regulator 1204 implanted between an artery 1250 and a vein 1252. The flow regulator 1204 can be manipulated so that high pressure does not continually flow through the graft 1200. For example, the flow regulator 1204 may at least partially constrain flow through the graft 1200 between dialysis treatments, and may allow high pressure flow through the graft 1200 during dialysis treatments. In some embodiments, high pressure flows through the graft 1200 less than about 20 hours per week, less than about 16 hours per week, less than about 12 hours per week, or less than about 10 hours per week. The reduction of pressure between treatments can reduce (e.g., minimize, eliminate) certain problems associated with venous barotrauma.

FIG. 13A illustrates a static graft 1390 implanted between an artery 1350 and a vein 1352. The graft 1390 allows continued high pressure fluid flow into the vein 1352. As needles from machines (e.g., needles 15, 16 from a hemodialysis machine 11 as illustrated in FIG. 10) are inserted into and removed from the graft 1390 a plurality of times every week, the material of the graft 1390 can become weakened in those regions, especially if accessed in approximately the same region each time. This can occur even in materials such as PTFE. As the material of the graft 1390 becomes weak, the continuous high pressure of the fluid in the graft 1390 from the artery 1350 can cause swelling or bulging in the weakened areas 1392 (e.g., similar to over inflation of a thin portion of a balloon). These pseudoaneurysms can cause patient discomfort, uneven fluid flow (e.g., eddies), thrombosis, massive bleeding from rupture, and/or can ultimately lead to failure of the graft 1390, which can necessitate emergent and multiple interventions to repair and salvage the graft. Ultimately, this may result in implantation of a new graft 1390 elsewhere in the patient's body (e.g., a different location on the appendage, a different appendage).

FIG. 13B illustrates an arterio-venous bridge graft 1300 comprising a flow regulator 1304 implanted between an artery 1350 and a vein 1352. The flow regulator 1304 can be manipulated so that high pressure does not continually flow through the graft 1300. For example, the flow regulator 1304 may at least partially constrain flow through the graft 1300 between dialysis treatments, and may allow high pressure flow through the graft 1300 during dialysis treatments. In some embodiments, high pressure flows through the graft 1300 less than about 20 hours per week, less than about 16 hours per week, less than about 12 hours per week, or less than about 10 hours per week. The reduction of pressure between treatments can reduce (e.g., minimize, eliminate) certain problems associated with portions of a graft 1300 weakened due to frequent usage. For example, the pressure may be reduced relative to the artery such that even the pressure proximal to the flow regulator 104 is insufficient to cause bulging. As described above, both of the needles 15, 16 may both be inserted distal to the flow regulator 104. In certain such embodiments, the weakened portions of the graft 1300 are both exposed to low pressure such that the pressure at the weakened portions is usually (i.e., between treatments) insufficient to cause pseudoaneurysm formation.

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the invention disclosed herein should not be limited by the particular disclosed embodiments described above. Although certain objects and advantages are described herein, not necessarily all such objects or advantages may be achieved in accordance with any particular embodiment, and other object and advantages are also possible. As an example, the grafts disclosed herein may decrease time to hemostasis and decrease blood loss when access needles are removed at the completion of a dialysis treatment, which may be achieved when a flow regulator is utilized to decrease flow through the remainder of the graft prior to withdrawing the access needles.

What is claimed is:

1. A method of regulating pressure in an arterio-venous bridge graft, the method comprising:
reversibly adjusting a cross-sectional area of the graft.

2. The method of claim 1, wherein reversibly adjusting the cross-sectional area comprises operating a flow regulator disposed between a first end and a second end of the graft.

3. The method of claim 2, wherein the flow regulator comprises a self-expanding stent substantially covered by polytetrafluoroethylene (PTFE).

4. The method of claim 2, wherein operating the flow regulator comprises manipulating a hollow member substantially surrounding a stent between a first position and a second position, the cross-sectional area of the stent configured to decrease upon movement of the hollow member from the first position to the second position and the cross-sectional area of the stent configured to increase upon movement of the hollow member from the second position to the first position.

5. The method of claim 2, wherein operating the flow regulator comprises increasing the cross-sectional area of the arterio-venous graft during a dialysis treatment and decreasing the cross-sectional area of the arterio-venous graft between dialysis treatments.

6. A method of fluidly coupling an artery and a vein, the method comprising:
connecting a first end of an implantable tubular member to the artery; and
connecting a second end of the implantable tubular member to the vein, the second end generally opposite the first end, a cross-sectional area of the implantable tubular member being reversibly adjustable.

7. The method of claim 6, wherein a flow regulator is disposed between the first end and the second end, the flow regulator configured to regulate fluid flow between the first end and the second end.

8. The method of claim 7, wherein the flow regulator comprises a self-expanding stent substantially covered by polytetrafluoroethylene (PTFE).

9. The method of claim 7, further comprising adjusting the cross-sectional area of the implantable tubular member by operating the flow regulator.

10. An arterio-venous graft comprising:
an implantable tubular member having a first end and a second end generally opposite the first end; and
a flow regulator between the first end and the second end, the flow regulator configured to reversibly regulate fluid flow between the first end and the second end.

11. The graft of claim 10, wherein the flow regulator comprises a mechanical switch.

12. The graft of claim 11, wherein the flow regulator comprises a hollow member substantially surrounding a stent, a cross-sectional area of the stent configured to decrease upon movement of the hollow member from a first position to a second position and configured to increase upon movement of the hollow member from the second position to the first position.

13. The graft of claim 10, wherein the flow regulator comprises a cylindrical flow limiter.

14. The graft of claim 10, wherein the flow regulator comprises an electronic switch.

15. The graft of claim 14, wherein the flow regulator comprises a timer configured to operate the switch.

16. The graft of claim 14, wherein the flow regulator comprises a sensor configured to operate the switch upon a change in a parameter of the fluid flow.

17. The graft of claim 16, wherein the parameter comprises at least one of fluid flowrate, fluid velocity, and fluid pressure.

18. The graft of claim 10, wherein the implantable tubular member has a length between the first end and the second end, wherein the flow regulator has a length extending along a longitudinal axis of the implantable tubular member, and wherein the length of the flow regulator is less than about ⅓ of the length of the implantable tubular member.

19. The graft of claim 10, wherein the flow regulator comprises a self-expanding stent substantially covered by polytetrafluoroethylene (PTFE).

20. The graft of claim 10, wherein the flow regulator further comprises a sleeve configured to prevent tissue ingrowth.
